(19) United States
(12) Patent Application Publication
(10) Pub. No.: US 2026/0199004 A1
(43) Pub. Date: Jul. 16, 2026

(54) BIPOLAR ARCHITECTURES FOR PFA CATHETERS WITH IRRIGATED ELECTRODE

(71) Applicant: Boston Scientific Scimed, Inc., Maple Grove, MN (US)

(72) Inventors: Nathan Paul Hagstrom, Blaine, MN (US); Michael Sean Coe, Plymouth, MN (US); Brendan Early Koop, Ham Lake, MN (US)

(21) Appl. No.: 19/444,894

(22) Filed: Jan. 9, 2026

Related U.S. Application Data
(60) Provisional application No. 63/744,062, filed on Jan. 10, 2025.

(51) Int. Cl. A61B 18/14; A61B 18/00

(52) U.S. Cl. CPC A61B 18/1492; A61B 2018/0022; A61B 2018/00267; A61B 2018/00351; A61B 2018/00577; A61B 2018/00613; A61B 2018/1472

(57) ABSTRACT

A catheter for ablating cardiac tissue through irreversible electroporation is disclosed. The catheter includes a shaft having a shaft proximal end, a shaft distal end, and a fluid tube extending from the shaft proximal end to the shaft distal end to receive a fluid. An electrode assembly extends from the shaft distal end. The electrode assembly includes splines defining an inner space and a flexible circuit having an ablation electrode disposed on the splines. A central post extends from the shaft distal end into the inner space and includes a post electrode and a fluid port disposed in the inner space. The fluid port is in fluid communication with the fluid tube to disperse the fluid into the inner space.

### CROSS REFERENCE TO RELATED APPLCATIONS

This application claims priority to U.S. Provisional Patent Application No. 63/744,062 entitled “BIPOLAR ARCHITECTURES FOR PFA CATHETERS WITH IRRIGATED ELECTRODE,” filed January 10, 2025, which is hereby incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. In RF ablation, a probe is inserted into the patient and radio frequency waves are transmitted through the probe to the surrounding tissue. The radio frequency waves generate heat, which destroys surrounding tissue and cauterizes blood vessels. Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

### SUMMARY

In Example 1, a catheter for ablating cardiac tissue through irreversible electroporation, the catheter comprising: a tubular outer shaft having a shaft proximal end and an opposite shaft distal end, the tubular outer shaft including a fluid tube extending from the shaft proximal end to the opposite shaft distal end configured to receive a fluid; an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising: a flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and a central post extending from the shaft distal end into the inner space having a post electrode and a fluid port disposed in the inner space, the fluid port in fluid communication with the fluid tube and configured to disperse the fluid into the inner space.

In Example 2, the catheter of Example 1, further comprising a plurality of spline sensing electrodes located on each spline.

In Example 3, the catheter of Example 2, wherein the plurality of spline sensing electrodes are included on the flex circuit.

In Example 4, the catheter of any of Examples 1-3, wherein the flex circuit comprises one ablation electrode.

In Example 5, the catheter of any of Examples 1-4, wherein the post electrode is generally planar.

In Example 6, the catheter of any of Examples 1-4, wherein the post electrode is generally bulbous.

In Example 7, the catheter of Example 6, wherein bulbous post electrode is porous and configured to disperse the fluid.

In Example 8, the catheter of Example 7, wherein the fluid port is disposed within the bulbous post electrode.

In Example 9, the catheter of any of Examples 6-8, wherein the bulbous post electrode is formed from a conductive fiber.

In Example 10, the catheter of any of Examples 1-9, wherein the fluid tube includes a plurality of branches, and each branch includes a corresponding fluid port configured to disperse fluid.

In Example 11, the catheter of any of Examples 1-10, wherein the dispersed fluid is configured to form a dispersed fluid region in contact with the post electrode.

In Example 12, the catheter of any of Examples 1-11, wherein the ablation electrode includes an exposed first surface area configured to deliver ablation energy and the dispersed fluid region in contact with the post electrode includes a second surface area configured to deliver ablation energy, wherein the second surface area is greater than the first surface area.

In Example 13, the catheter of any of Examples 1-14, further comprising a hub sensing electrode centrally located on the central hub portion of the electrode assembly.

In Example 14, the catheter of any of Examples 1-13, wherein the fluid has a higher conductivity than the cardiac tissue.

In Example 15, the catheter of any of Examples 1-14, wherein the ablation electrode is configurable as one of a cathode and an anode and the post electrode is configurable as the other of the anode and the cathode in a bipolar mode.

In Example 16, a catheter for ablating cardiac tissue through irreversible electroporation, the catheter comprising: a tubular outer shaft having a shaft proximal end and an opposite shaft distal end, the tubular outer shaft including a fluid tube extending from the shaft proximal end to the opposite shaft distal end configured to receive a fluid; an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising: a flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and a central post extending from the shaft distal end into the inner space having a post electrode and a fluid port disposed in the inner space, the fluid port in fluid communication with the fluid tube and configured to disperse the fluid into the inner space.

In Example 17, the catheter of Example 16, further comprising a plurality of spline sensing electrodes located on each spline.

In Example 18, the catheter of Example 17, wherein the plurality of spline sensing electrodes are included on the flex circuit.

In Example 19, the catheter of Example 16, wherein the flex circuit comprises one ablation electrode.

In Example 20, the catheter of Example 16, wherein the post electrode is generally planar.

In Example 21, the catheter of Example 16, wherein the post electrode is generally bulbous.

In Example 22, the catheter of Example 21, wherein bulbous post electrode is porous and configured to disperse the fluid.

In Example 23, the catheter of Example 22, wherein the fluid port is disposed within the bulbous post electrode.

In Example 24, the catheter of Example 21, wherein the bulbous post electrode is formed from a conductive fiber.

In Example 25, the catheter of Example 16, wherein the fluid tube includes a plurality of branches, and each branch includes a corresponding fluid port configured to disperse fluid.

In Example 26, the catheter of Example 16, wherein the dispersed fluid is configured to form a dispersed fluid region in contact with the post electrode.

In Example 27, the catheter of Example 16, wherein the ablation electrode includes an exposed first surface area configured to deliver ablation energy and the dispersed fluid region in contact with the post electrode includes a second surface area configured to deliver ablation energy, wherein the second surface area is greater than the first surface area.

In Example 28, the catheter of Example 16, further comprising a hub sensing electrode centrally located on the central hub portion of the electrode assembly.

In Example 29, the catheter of Example 16, wherein the fluid has a higher conductivity than the cardiac tissue.

In Example 30, the catheter of Example 16, wherein the ablation electrode is configurable as one of a cathode and an anode and the post electrode is configurable as the other of the anode and the cathode in a bipolar mode.

In Example 31, a catheter for ablating cardiac tissue through irreversible electroporation, the catheter comprising: a tubular outer shaft having a shaft proximal end and an opposite shaft distal end, the tubular outer shaft including a fluid tube extending from the shaft proximal end to the opposite shaft distal end configured to receive a fluid; an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising: an flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and a central post extending from the shaft distal end into the inner space having a post electrode and a fluid port disposed in the inner space, the fluid port in fluid communication with the fluid tube and configured to disperse the fluid into the inner space, the central post proximal to the proximal end of the ablation electrode.

In Example 32, the catheter of Example 31, wherein the ablation electrode is configurable as one of a cathode and an anode and the post electrode is configurable as the other of the anode and the cathode in a bipolar mode.

In Example 33, the catheter of Example 32, wherein the dispersed fluid is configured to form a dispersed fluid region in contact with the post electrode.

In Example 34, a catheter for ablating cardiac tissue through irreversible electroporation, the catheter comprising: a tubular outer shaft having a shaft proximal end and an opposite shaft distal end, the tubular outer shaft including a fluid tube extending from the shaft proximal end to the opposite shaft distal end configured to receive a fluid; an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising: a flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and a central post extending from the shaft distal end into the inner space having a post electrode and a fluid port disposed in the inner space, the fluid port in fluid communication with the fluid tube and configured to disperse the fluid into the inner space, the central post proximal to the proximal end of the ablation electrode, wherein the post electrode is one of a planar electrode or a bulbous electrode.

In Example 35, the catheter of Example 34, wherein the post electrode is a bulbous electrode, and the bulbous electrode is inflatable.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2A is a perspective illustration of a distal portion of a splined catheter for use in the electrophysiology system of FIG. 1, in accordance with embodiments of the subject matter of the disclosure.

FIGS. 2B is an end view illustration of the distal portion of the splined catheter of FIG. 2A.

FIG. 2C is a partial plan view an electrode assembly of the splined catheter of FIG. 2A as shown in two-dimensions, in accordance with embodiments of the subject matter of the disclosure.

FIG. 2D is an enlarged plan view of an outwardly facing portion of a spline of the electrode assembly shown in FIGS. 2A-2C, in accordance with embodiments of the subject matter of the disclosure.

FIG. 2E is a schematic cross-sectional view of a configuration of a spline of the electrode assembly of FIG. 2A, in accordance with embodiments of the subject matter of the disclosure.

FIG. 3A is a schematic partial side view of a first embodiment an electrode assembly shown in FIG. 2A, in accordance with embodiments of the subject matter of the disclosure.

FIG. 3B is a schematic partial side view of a second embodiment an electrode assembly shown in FIG. 2A, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

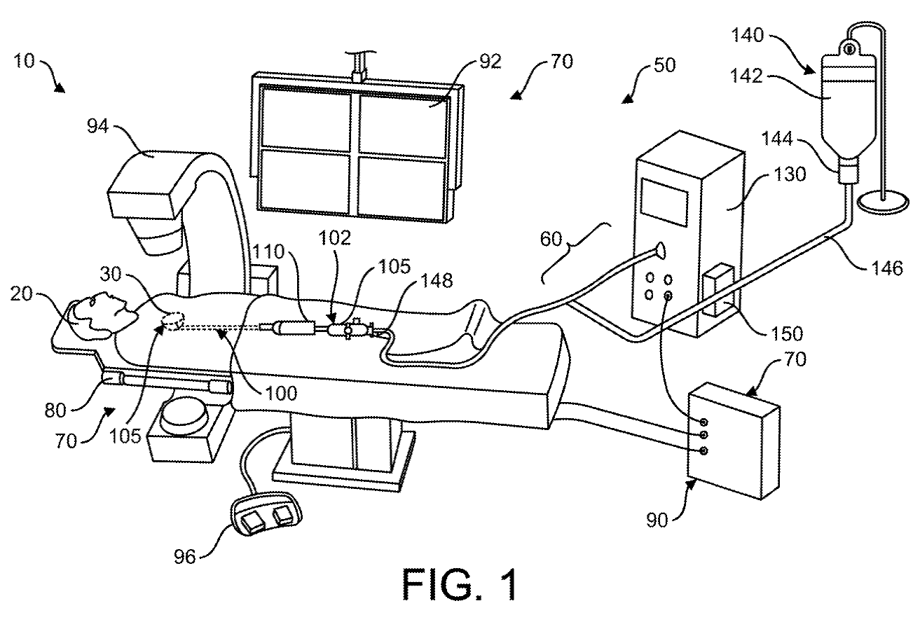

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1. Other arrangements of connecting elements, including wireless connecting elements, are contemplated.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient’s heart 30. Access to the patient’s heart can be obtained through a vessel, such as a peripheral artery or vein. Once access to the vessel is obtained, the electroporation catheter 100 can be navigated to within the patient’s heart, such as within a chamber of the heart. In embodiments, the electroporation catheter system 60 is configured to deliver electric field energy to targeted tissue in the patient’s heart 30 to create tissue apoptosis, rendering the tissue incapable of conducting electrical signals.

The example electroporation catheter 100 includes an elongated catheter shaft and distal portion 105 configured to be deployed proximate target tissue, such as within a chamber of the patient’s heart. The distal portion 105 may include a basket, balloon, spline, configured tip, or other electrode deployment mechanism to effect treatment. The electrode deployment mechanism includes an electrode assembly, or array, comprising of an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 100, such as a plug configured to be mechanically and electrically coupled to the electroporation console 130, for example, either directly or via intermediary electrical conductors such as cabling. In one example, the electroporation console 130 is configured to provide an electrical signal, such as a plurality of concurrent or space-apart-time electrical signals, to the electrically connected electroporation catheter 100 along lead conductors to the spaced-apart electrodes. The spaced-apart electrodes are configured to generate a selected electrical field proximate the target tissue, based on the electrical signals from the electroporation console 130, to effect electroporation.

A selected electrical field can be generated with the electrodes to effect electroporation. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 100. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 100 can be operated in a selected mode such as monopolar mode or bipolar mode. During monopolar operation of the catheter 100, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 100, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient’s body, but rather through a localized portion of tissue proximate the electrode assembly.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the OPAL™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

In embodiments, the electroporation catheter 100 is used in combination with a fluid source 140. Fluid source 140 may comprise a bag of fluid from which fluid 142 may flow through a drip chamber 144, to delivery tubing 146 and to the electroporation catheter 100. In some embodiments, the proximal portion 102 of the electroporation catheter 100 includes a fluid system having a proximal fluid port 148 configured to be connected in fluid communication with the delivery tubing 146 and to receive the fluid 142 from the fluid source 140. The fluid system of the electroporation catheter 100 is configured to deliver the fluid received at the fluid port 148 to the distal portion 104 of the electroporation catheter 100. In some embodiments, the electroporation catheter 100 includes delivery tubing extending along the elongate shaft from the proximal fluid port 148 at the proximal portion 102 to the distal portion 105 and configured to be in fluid communication with the fluid source 140 to disperse fluid 142 via a distal fluid port proximate to the electrode assembly.

In one example, the fluid 142 includes saline and can include physiologic saline such as sodium chloride (NaCl) 0.9% weight/volume solution. Saline is an electrically conductive fluid, and other suitable electrically conductive fluids can be used. In other examples, the fluid may include a nonconductive fluid, such as deionized water. The fluid delivery tubing 146 in the example passes through pump 150 to convey fluid to the electroporation catheter 100 and control fluid flow. Pump 150 in some embodiments is a peristaltic pump such as a rotary peristaltic pump or a linear peristaltic pump. A peristaltic pump conveys the fluid through the delivery tubing 146 by way of intermittent forces placed on the external surface of the delivery tubing 146, and the mechanical elements of the pump 150 places forces on the external surface of the delivery tubing 146 so as not come into direct contact with the fluid 142, which can reduce the likelihood of fluid contamination. some embodiments of clinical setting 10 do not include a pump, and fluid is provided to the electroporation catheter 100 via gravity.

The pump 150, electroporation catheter system 60 or other system is configured to automatically determine the fluid flow rate, manually allow a clinician to select the fluid flow rate, or some combination of such as to allow the clinician to vary the fluid flow rate within range of fluid flow rates or during operation of the electroporation catheter such as while delivering ablation energy. While not being bound to a particular theory, the relationship between the variables of fluid flow rate Q (such as in units of cubic centimeters per minute (cc/min)) and RF power setting PS (such as in units of watts) can be configured to promote an efficient dispersion of ablation energy at electrode assembly as well as to inhibit undesired effects such as disperse to much ablation energy or otherwise negatively affect a pulsed electrical field. The electroporation console 130 can be configured to include control of the pump 150. In this example, the speed of the pump 150, and the fluid throughput, can be predetermined based on input variables such as electrical field strength and the fluid flow rate setting. In one example, the pump 150 can be integrated with the electroporation console 130.

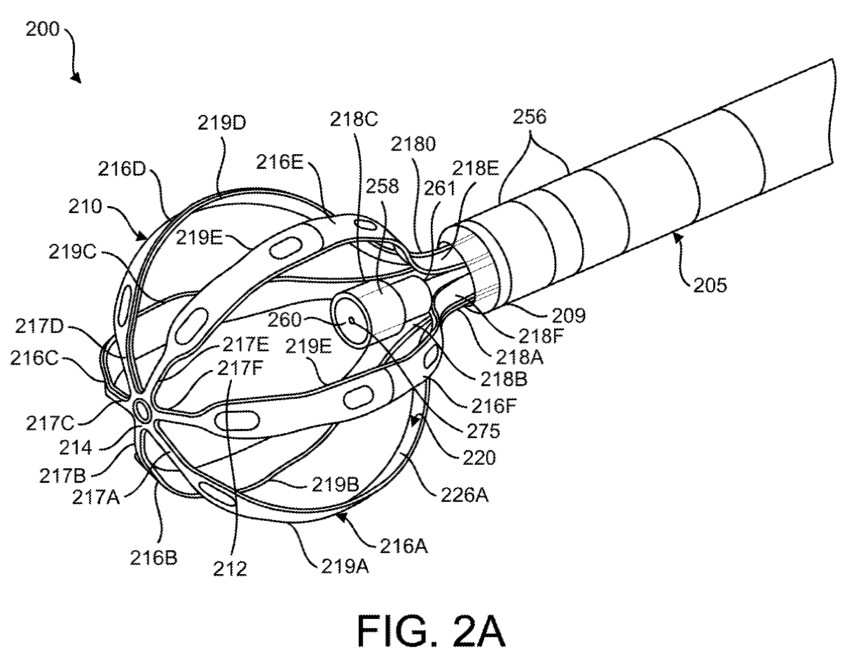

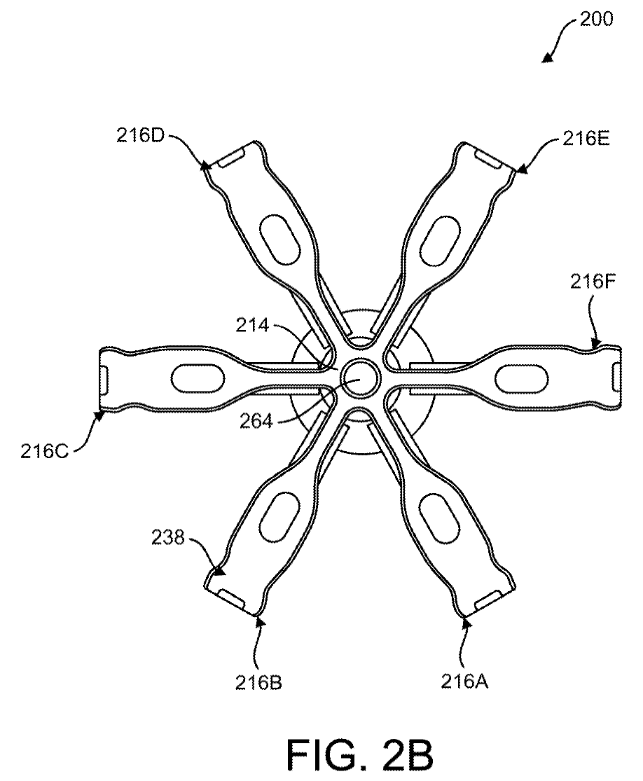

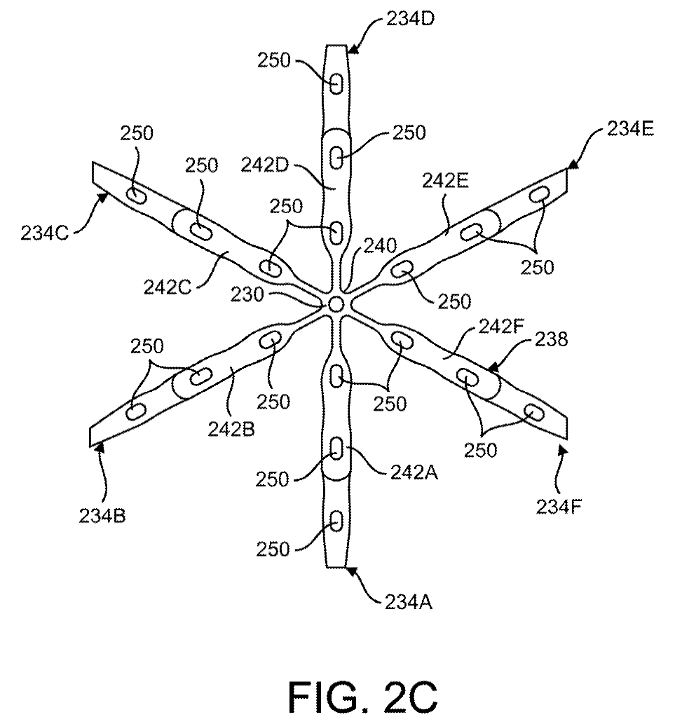

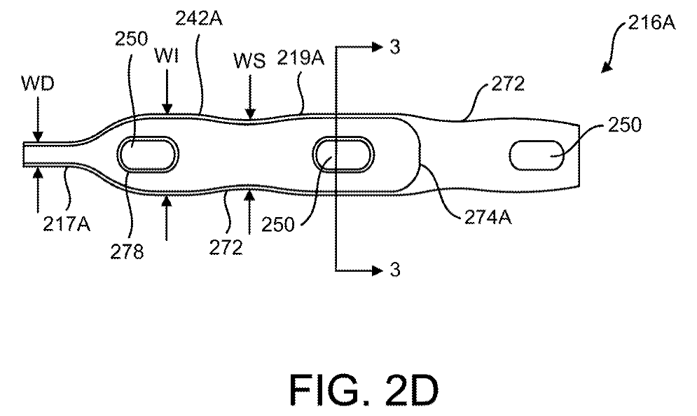

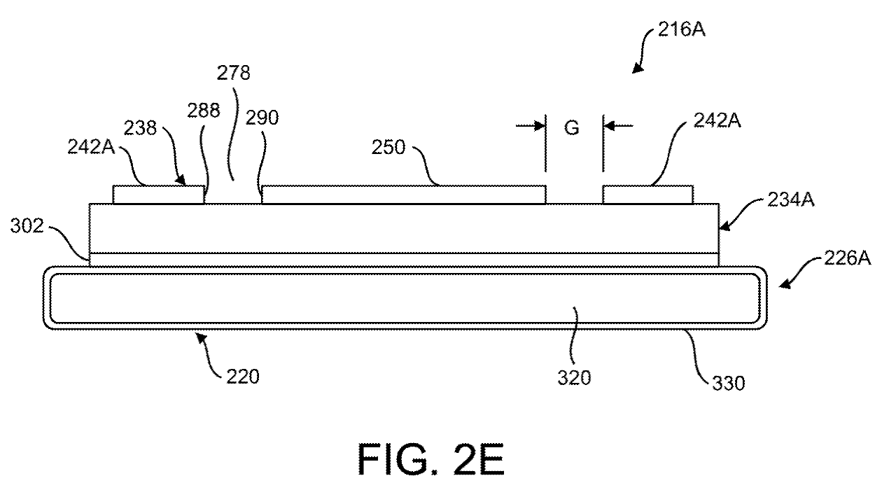

FIGS. 2A and 2B are partial perspective and end view illustrations, respectively, of an electroporation catheter 200 having a catheter distal portion 205 according to an embodiment of the present disclosure. The electroporation catheter 200 corresponds to the electroporation catheter 100 described with respect to FIG. 1. The electroporation catheter 200 has a tubular outer shaft 202 having a shaft distal end 209, and an electrode assembly 210 extending distally from the distal end 209 of the outer shaft 202. In embodiments, the electrode assembly 210 is configured to self-expand from a collapsed configuration when constrained within a delivery sheath to a pre-defined expanded configuration defining an inner space 212. The electrode assembly 210 comprises an ablation electrode configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

Overall, the electrode assembly 210 and other electrode assembly embodiments described herein within the scope of the present disclosure, is primarily designed for the creation of relatively localized ablation lesions (i.e., focal lesions), as compared to relatively large diameter circumferential lesions created in pulmonary vein isolation procedures. However, the skilled artisan will appreciate that the teachings of the present disclosure can be readily adapted for a catheter capable of large diameter circumferential lesions. The designs of the various electrode assembly embodiments described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

In one embodiment, the electrode assembly 210 is operated in a bipolar mode. The ablation electrode is configured as a plurality of electrodes in which at least one ablation electrode is configurable as one of a cathode and an anode and the at least one other ablation electrode is configurable as the other of the anode and the cathode to generate the pulsed electric fields. For example, one or more ablation electrodes are configurable as a cathode, or active electrode, and one or more other ablation electrodes are configurable as an anode, or return electrode. As understood by those skilled in the art, an ablation electrode configured as a cathode in one pulse of a biphasic waveform is configured as an anode in another pulse of the biphasic waveform, and the other ablation electrode configured as the anode in the one pulse is configured as the cathode in the another pulse. Electrode assemblies operated in bipolar mode can provide advantages such as effective therapy via local energy delivery. Local energy delivery via the active electrode and return electrode proximate the target site can result in lower muscle stimulation over monopolar configurations. Further, the shape, relative positions of the cathode and anode electrodes, and relative amounts of exposed, or effective, surface areas of the electrodes are applied to direct the electric fields in a manner suitable for the electrode assembly. For example, electric fields can be pushed in a vector toward the cathodic electrodes to focus therapy.

In some examples of electrode assemblies having a bipolar configuration, a first ablation electrode or first set of ablation electrodes, configurable as a cathode, is located on a basket of splines extending distally from a shaft, and a second ablation electrode or second set of ablation electrodes, configurable as the anode, are located on the shaft as ring electrodes proximal to the basket of splines. Such configurations provide for effective, focused therapy and low muscle stimulation over monopolar configurations. But such configurations also reduce usability as the sheath is required to be fully retracted over the shaft ring electrodes for use in a bipolar mode. Additionally, the spacing between the first set of ablation electrodes and the shaft ring electrodes generates a relatively large electric field causing a relatively large lesion in the target tissue, which reduces an ability of a clinician to direct or control the location of therapy.

In the embodiments of electrode assemblies 210, the anodic elements are more distal than with other electrode assemblies and also retain an electrode surface area ratio with the cathodic elements to push the electric field toward the cathode to maintain effective therapy. Applicants have discovered that electrode surface area is analogous to electrochemical surface impedance of the electrode. The electrode assembly 210 modifies impedance directly surrounding the surface area of the anode to provide increased flexibility in the size and shape of the anode and the placement of the anode.

FIG. 2C is a partial plan view of the electrode assembly 210 of the electroporation catheter 200 shown, shown in two-dimensions to illustrate the layout of the electrode assembly 210. Referring to FIGS. 2A-2C together, in the illustrated embodiment, the electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown, in the illustrated embodiment, the intermediate portion 219A-219F of each spline 216A-216F has a lateral width that is greater than the lateral width of each of the respective distal end portions 217A-217F. In embodiments, the particular geometry of the splines 216A-216F and the related components, e.g., ablation and mapping electrodes, is optimized to provide desired mechanical and therapeutic/diagnostic capabilities.

In the illustrated embodiment, the splines 216A-216F are composed of a support member 220 and a flexible circuit 222 secured to and disposed over an outer surface of the support member 220. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In embodiments, the support member 220 is formed from a superelastic metal alloy, e.g., a nickel-titanium alloy.

The support member 220 includes a support member hub 224 and a plurality of support member branches (for ease of illustration, only support member branch 226A is labeled in FIG. 2A). In embodiments, the support member branches are integrally formed with and extend proximally from the support member hub 224. For example, the entire support member 200 may be cut from a single sheet of material using conventional manufacturing techniques. This unitary structure provides robust structural properties, for example, selective flexibility and enhanced fatigue characteristics, particularly in areas that are subject to relatively high stresses during manufacture and use of the electroporation catheter 200. Forming the support member 220 from a superelastic material such as a nickel-titanium alloy facilitates configuring the support member 220 to assume its desired unconstrained shape such as shown in FIG. 2A due to the shape memory properties of the material, while providing sufficient flexibility necessary to collapse the electrode assembly 210 within a delivery sheath. In embodiments, the support member branches can be selectively configured along their lengths to tune the mechanical characteristics of the electrode assembly 210.

The flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches. The flexible circuit 222 comprises a layered construction including one or more dielectric substrate layers, and conductive traces formed thereon. Similar to the support member 220, the unitary construction of the flexible circuit 222 enhances its structural properties, for example, by minimizing joints or other discontinuities at regions subject to relatively high stresses during use.

As shown, the flexible circuit 222 includes an ablation electrode 238 that has an ablation electrode hub portion 240 and a plurality of ablation electrode branches 242A-242F. In the illustrated embodiment, the distal ablation electrode hub portion 240 is located on the flex circuit hub 230. Additionally, the ablation electrode branches 242A-242F are integrally formed with the ablation electrode hub portion 240. Each of the ablation electrode branches 242A-242F extends proximally along a portion of a respective one of the flex circuit branches 234A-234F.

As further shown, the flexible circuit 222 includes a plurality of spline sensing electrodes 250. In the illustrated embodiment, two of the spline sensing electrodes 250 are disposed within a periphery of each of the ablation electrode branches 242A-242F, and one of the spline sensing electrodes 250 is located proximal to each of the ablation electrode branches 242A-242F on a respective flex circuit branch 234A-234F. The illustrated configuration is exemplary only, and other embodiments of the catheter 200 may have alternative configurations. Thus, in various embodiments, one or more of the spline sensing electrodes 250 may be disposed within the periphery of one or more of the ablation electrode branches 242A-242F and electrically isolated therefrom, and one or more of the spline sensing electrodes 250 may be located proximal to the ablation electrode branches 242A-242F on the respective flex circuit branch 234A-234F. In still other embodiments, no spline sensing electrodes 250 may be located outside the peripheries of the ablation electrode branches 242A-242F.

In some embodiments, the structural functionality of the support member 220 can be provided by a suitably designed flexible circuit 222. As such, although the electrode assembly 210 is described in detail as including the support member 220 as a primary structural member, in other embodiments the support member 220 can be omitted in its entirety and the corresponding functionality can be provided by the flexible circuit 222.

In the particular illustrated embodiment, the electroporation catheter 200 includes a pair of shaft electrodes 256 located proximate the distal end 209 of the outer shaft 202, In the illustrated embodiment, the electrode assembly 210 further includes a hub sensing electrode 264 centrally located on the flex circuit hub 230. In embodiments, one or both of the shaft electrodes 256 can be configured to be paired with the ablation electrode 238 to form an anode/cathode ablation electrode pair for generation of an ablative electric field in a bipolar mode in certain applications. In embodiments, the shaft electrodes 256 may have additional functions, e.g., and without limitation, as additional sensing electrodes for sensing cardiac electrical signals, and for use as localization sensors for impedance tracking of the electrode assembly 210. In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

FIG. 2D is an enlarged plan view of a portion of the spline 216A, the ablation electrode branch 242A, and the flex circuit branch 234A, according to embodiments of the present disclosure. The structural features illustrated in FIG. 2D are representative the splines 216A-216F, the ablation electrode branches 242A-242F and the flex circuit branches 234A-234F.

The distal end portion 217A of the spline 216A has a maximum width WD, and the intermediate portion 219A of the spline 216A has a maximum width WI that is greater than the maximum width WD of the distal end portion. In the particular embodiment shown, the intermediate portion 219A further includes one or more scalloped regions 272 wherein the opposing outer edges of the spline 216A have a concave shape. In embodiments, the scalloped regions 272 are selectively located along the length of the spline 216A and each have a scalloped region minimum width WS that is less than the maximum width WI of the intermediate portion 219A. When present, the scalloped regions 272 affect the mechanical properties (e.g., bending flexibility) of the spline 216A, to, for example, facilitate deformation of the spline 216A when it is in contact with target tissue, as well as facilitating collapse of the electrode assembly 210 when it is retracted into a delivery sheath. However, in some embodiments, the scalloped regions 272 are omitted, and the spline 216A has a generally linear shape along the intermediate portion 219A. In the illustrated embodiment, at least one of the scalloped regions 272 is located in the region of the spline 216A on which a portion of the ablation electrode branch 242A is disposed, and between the spline sensing electrodes 250 located thereon. The ablation electrode branch 242A has a proximal end 274A. In the illustrated embodiment, the proximal end 274A is contoured and shaped to enhance electric field generation and clinical efficacy when the catheter 200 is configured to operate in bi-polar energy delivery mode, with the ablation electrode 238 and one or both of the shaft electrodes 256 paired as a bi-polar electrode pair. In other embodiments, however, the proximal end 274A can take on different shapes, e.g., semi-circular. The location of the proximal end 274A (which as will be appreciated, defines the length of the ablation electrode branch 242A and consequently defines, in part, the overall surface area of the ablation electrode 238) can be varied from embodiment to embodiment depending on the particular clinical needs required of the catheter 200. The ablation electrode branch 242A includes a plurality of ablation electrode branch apertures 278, and one of the spline sensing electrodes 250 is disposed within each of the ablation electrode branch apertures 278.

Application of high voltage pulsed field ablation energy to the ablation electrode 238 creates a high strength electrical field. The support member 220 is disposed in the high strength electrical field. In cases in which a conductive material is used as a stiffener in the support member 220, undesirable electrical coupling (via capacitance or some other mechanism) between the conductive support member 220 and the flex circuit may occur. Such electrical coupling can result in localized heating of the ablation electrode 238 and the sense electrodes 250. Accordingly, electrical coupling between the flex circuit and the conductive support member 220 is to be avoided to maintain a viability of the flexible-circuit-based electroporation catheter architecture.

FIG. 2E is a schematic cross-sectional view of the spline 216A taken along the line 2E-2E in FIG. 2D, illustrating an exemplary configuration of the flex circuit branch 234A disposed on the support member branch 226A on the spline 216A. In embodiments, the particular design of the flex circuit branch 234A (and the flex circuit as a whole) can be tailored for the particular clinical needs present. In the particular embodiment illustrated in FIG. 2E, the flex circuit branch 234A is secured to the support member branch 226A by an adhesive layer 302, which may be any suitable adhesive.

FIG. 2E illustrates the ablation electrode 238 and the spline sensing electrode 250 are disposed on an upper surface of the flex circuit branch 234A. In embodiments, both the ablation electrode and the sensing may have a coating of a suitable biocompatible metal, e.g., gold. In embodiments, the outer surfaces of the electrodes may be treated to provide the electrical properties desired for the particular clinical application. The proximal ablation electrode aperture 278 is bounded by an inner peripheral surface 288 of the ablation electrode branch 242A, and an outer peripheral surface 290 of the spline sensing electrode 250 is spaced from the inner peripheral surface 288 of the ablation electrode branch 242A by a gap G. In some embodiments, the gap G and portions of the ablation electrode 238 and the spline sensing electrode 250 may be selectively covered by a dielectric material (not shown).

FIG. 2E further illustrates one embodiment of a support member 220 configured to reduce the likelihood of electrically coupling between the support member 220 and the flex circuit branch 234A. The support member 220, as illustrated via support member branch 226A, includes an electrically conductive base member 320 covered with an electrically insulative coating 330. In the illustrated example, the electrically insulative coating 330 is a thin film of a dielectric material such as silicone, parylene, polyvinylidene fluoride, or other materials having similar dielectric properties. In one embodiment, the electrically insulative coating 330 is deposited on the base member 320 via an appropriate process including spay coat, dip coat, chemical vapor deposition, and atomic layer deposition, and the like. In one embodiment, the electrically insulative coating 330 encapsulates the entire electrically conductive base member 320 distal to the shaft distal end 209 (see FIG. 2A). The thicknesses of the conductive base member 320 and the coating 330 may be selectively tailored to provide a desired degree of structural support and the aforementioned electrical decoupling. In one exemplary embodiment, the base member 320 may have a thickness of about 68 micrometers, and the dielectric coating 330 may have a thickness of about 12 micrometers, such that the overall thickness of the support member branch 226A is about 92 micrometers.

Returning to FIG. 2A, the electrode assembly 210 includes a central post 258 extending distally from the distal end 209 of the outer shaft 202. The central post 258 extends partially into the inner space 212 and includes a post electrode 260. In embodiments, the central post 258 may house additional components. For example, in embodiments, a magnetic navigation sensor (not shown) may be partially or wholly disposed within the central post 258. However, in other embodiments such a sensor may be located elsewhere on the electroporation catheter 200 (e.g., within the outer shaft 202). In some embodiments, the central post 258 includes a distal fluid port 275 in fluid communication with proximal fluid port 148 and configured to disperse fluid 142 from fluid source 140. In some embodiments, the catheter shaft 202 includes a flexible fluid tube therein extending from the proximal portion 102 of the catheter 100, 200 to the distal portion 205 of the shaft 202 and terminating at the distal fluid port 275. The fluid tube is in fluid communication with the proximal fluid port 148 to receive fluid 142. Fluid 142 is transported along the fluid tube to the distal fluid port 275.

The electrode assembly 210 is configured to operate in bipolar energy delivery mode, with the ablation electrode 238 and post electrode 260 paired as a bipolar electrode pair. In one embodiment, the ablation electrode 238 is configured as the cathode, and the post electrode 260 is configured as the anode. The dispersal of fluid from the distal fluid port 275 proximate the post electrode 260 creates a fluid dispersal region that modifies impedance directly surrounding the surface area of the anode. In this embodiment, an ablation field vector is directed from the cathode to the anode, or from outside the electrode assembly 210 via the outwardly facing ablation electrode 238 to the post electrode 260 and fluid dispersal region within the inner space 212. In embodiments in which the fluid 142 is of a higher conductivity than surrounding fluid, such as the body fluid proximate the target tissue and the ablation electrode 238, the dispersed fluid effectively creates a larger electrode, or anode, than the surface area of the post electrode 260. For instance, fluid dispersal region proximate the distal fluid port 275 in contact with the post electrode 260 forms an effective anode.

The fluid dispersed from the center post 258 creates an effective overall surface area of the anode that can be varied by the amount of fluid dispersed. With no fluid dispersed, the effective area of the anode is the surface area of the post electrode 260; with a little fluid dispersed, the effective area of the anode is larger than the surface of the post electrode 260; and with a greater amount of fluid dispersed, the effective area of the anode is even larger than the effective area of the anode with a little fluid dispersed. Accordingly, the effective area of the anode is controlled via the amount of the fluid dispersed when the bipolar energy is delivered. The effective area of the anode can be varied based on the particular clinical needs required of the catheter 200. The amount of fluid dispersal can be based on design concerns such as the total surface area of the ablation electrode 238 and the effective area of the anode with fluid dispersal. In one embodiment, the outwardly facing ablation electrode 238 includes an exposed first surface area configured to deliver ablation energy and the fluid dispersing center post 258 includes an effective second surface area configured to deliver ablation energy, wherein the second surface area is greater than the first surface area. In another embodiment, the first and second surface areas are equal.

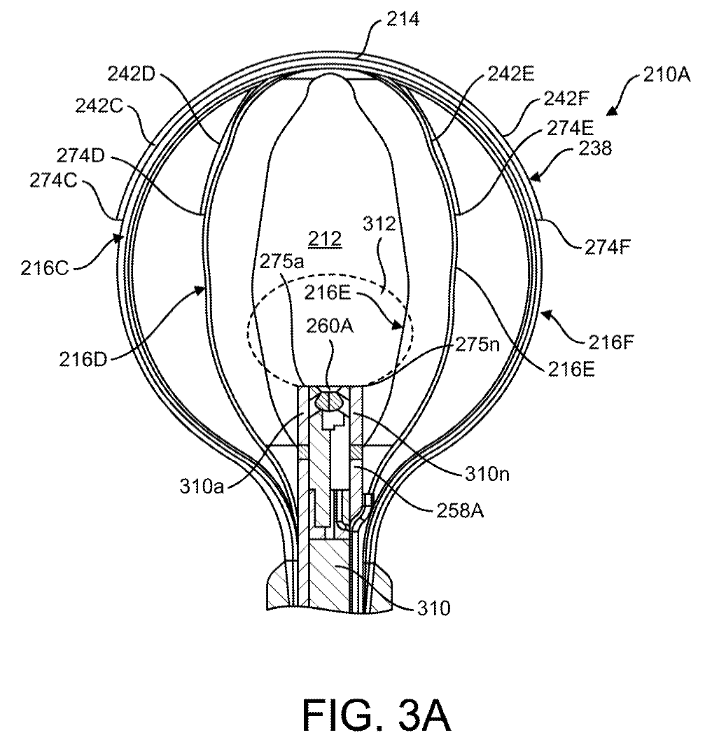

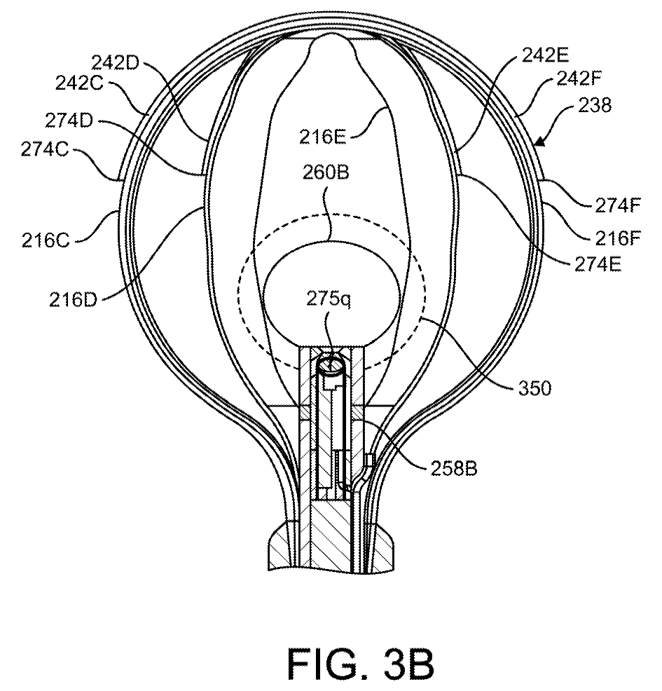

FIG. 3A is a schematic partial side view of a first embodiment an electrode assembly 210A corresponding with the electrode assembly 210 shown in FIG. 2A. Electrode assembly 210A as a whole has the distally-located central hub portion 214 and the plurality of splines 216A-216F extending proximally from the central hub portion 214 as described above, but with splines 216A, 216B removed for clarity to illustrate center post 258A and post electrode 260A. In the illustrated embodiment, the center post 258A includes a plurality of distal fluid port 275a…275n, disposed around the periphery of post electrode 260A. A fluid delivery tube 310 extends along the shaft 202, such as from the proximal portion 102 to the distal portion 205, and includes branches 310a…310n, which are associated with distal fluid ports 275a…275n. Fluid ports 275a…275n are in fluid communication with proximal fluid port 148 at the proximal portion of fluid delivery tube 310, and are configured to disperse fluid received at proximal fluid port 148 into the inner space 212 to create a fluid dispersal region 312. The fluid dispersal region 312 is in contact with the post electrode 260A. When the post electrode 260A and ablation electrode 238 are energized in with bipolar energy, the fluid dispersal region creates an effective anode for the electric field. In the illustrated embodiment, the center post 258 terminates proximally from the proximal ends 274A-274F of the ablation electrode 238 with respect to the longitudinal axis of the catheter 200.

FIG. 3B is a schematic partial side view of a second embodiment an electrode assembly 210B corresponding with the electrode assembly 210 shown in FIG. 2A. Electrode assembly 210A as a whole has the distally-located central hub portion 214 and the plurality of splines 216A-216F extending proximally from the central hub portion 214 as described above, but with splines 216A, 216B removed for clarity to illustrate center post 258B and bulbous post electrode 260B. In the illustrated embodiment, the center post 258B includes a distal fluid port 275q, disposed within the bulbous post electrode 260B. The fluid delivery tube 310 extends along the shaft 202, such as from the proximal portion 102 to the distal portion 205, and is associated with distal fluid port 275q. Fluid port 275q is in fluid communication with proximal fluid port 148 at the proximal portion of fluid delivery tube 310, and is configured to disperse fluid received at proximal fluid port 148 into the bulbous post electrode 260B. In some embodiments, the bulbous post electrode 260B is configured as an inflatable balloon having a first, or deflated, and a second, or inflated configuration as shown in FIG. 3B. The bulbous electrode 260B can be placed in the deflated configuration in circumstances in which the splines 216A-216F are in the collapsed configuration, such as when constrained within the delivery sheath. The bulbous post electrode 260B can be placed in the inflated configuration in circumstances in which the splines 216A-216F are in the expanded configuration, such as to receive pulsed electrical signals/waveforms from the electroporation console 130. The bulbous post electrode 260B in the inflated configuration occupies a portion of the inner space 212. In the illustrated embodiment, the bulbous post electrode 260B in the inflated configuration terminates proximally from the proximal ends 274A-274F of the ablation electrode 238 with respect to the longitudinal axis of the catheter 200. In some embodiments, the bulbous post electrode 260B transitions to the inflated configuration when filled with the fluid 142, via a self-expanding material, or via a transitioning mechanism operated by a clinician.

In the illustrated embodiment, the bulbous post electrode 260B is porous in various places around the outer surface so as to disperse the fluid from distal fluid port 275q into the inner space 212 to create a fluid dispersal region 350. In one example, the porous bulbous post electrode 260B is includes an electrically conductive surface having a plurality of holes configured to disperse fluid. In some embodiments, the porous electrically conductive surface is formed from conductive fibers. In one embodiment, the electrode 260B is formed from electronic spun conductive fibers. Electrospinning is a fiber production method for producing the electrically conductive surface of the post electrode 260B. The fibrous post electrode is configured to disperse fluid through the holes in the fibrous material. The fluid dispersal region 330 is in contact with the electrically conductive bulbous post electrode 260A. When the post electrode 260A and ablation electrode 238 are energized in with bipolar energy, the fluid dispersal region creates an effective anode for the electric field. In the illustrated embodiment, the fluid dispersal region 350 terminates proximally from the proximal ends 274A-274F of the ablation electrode 238 with respect to the longitudinal axis of the catheter 200.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablating cardiac tissue through irreversible electroporation, the catheter comprising:
a tubular outer shaft having a shaft proximal end and an opposite shaft distal end, the tubular outer shaft including a fluid tube extending from the shaft proximal end to the opposite shaft distal end configured to receive a fluid;
an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising:
a flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and
a central post extending from the shaft distal end into the inner space having a post electrode and a fluid port disposed in the inner space, the fluid port in fluid communication with the fluid tube and configured to disperse the fluid into the inner space.

2. The catheter of claim 1, further comprising a plurality of spline sensing electrodes located on each spline.

3. The catheter of claim 2, wherein the plurality of spline sensing electrodes are included on the flex circuit.

4. The catheter of claim 1, wherein the flex circuit comprises one ablation electrode.

5. The catheter of claim 1, wherein the post electrode is generally planar.

6. The catheter of claim 1, wherein the post electrode is generally bulbous.

7. The catheter of claim 6, wherein bulbous post electrode is porous and configured to disperse the fluid.

8. The catheter of claim 7, wherein the fluid port is disposed within the bulbous post electrode.

9. The catheter of claim 6, wherein the bulbous post electrode is formed from a conductive fiber.

10. The catheter of claim 1, wherein the fluid tube includes a plurality of branches, and each branch includes a corresponding fluid port configured to disperse fluid.

11. The catheter of claim 1, wherein the dispersed fluid is configured to form a dispersed fluid region in contact with the post electrode.

12. The catheter of claim 1, wherein the ablation electrode includes an exposed first surface area configured to deliver ablation energy and the dispersed fluid region in contact with the post electrode includes a second surface area configured to deliver ablation energy, wherein the second surface area is greater than the first surface area.

13. The catheter of claim 1, further comprising a hub sensing electrode centrally located on the central hub portion of the electrode assembly.

14. The catheter of claim 1, wherein the fluid has a higher conductivity than the cardiac tissue.

15. The catheter of claim 1, wherein the ablation electrode is configurable as one of a cathode and an anode and the post electrode is configurable as the other of the anode and the cathode in a bipolar mode.

16. A catheter for ablating cardiac tissue through irreversible electroporation, the catheter comprising:
a tubular outer shaft having a shaft proximal end and an opposite shaft distal end, the tubular outer shaft including a fluid tube extending from the shaft proximal end to the opposite shaft distal end configured to receive a fluid;
an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising:
a flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and
a central post extending from the shaft distal end into the inner space having a post electrode and a fluid port disposed in the inner space, the fluid port in fluid communication with the fluid tube and configured to disperse the fluid into the inner space, the central post proximal to the proximal end of the ablation electrode.

17. The catheter of claim 16, wherein the ablation electrode is configurable as one of a cathode and an anode and the post electrode is configurable as the other of the anode and the cathode in a bipolar mode.

18. The catheter of claim 17, wherein the dispersed fluid is configured to form a dispersed fluid region in contact with the post electrode.

19. A catheter for ablating cardiac tissue through irreversible electroporation, the catheter comprising:
a tubular outer shaft having a shaft proximal end and an opposite shaft distal end, the tubular outer shaft including a fluid tube extending from the shaft proximal end to the opposite shaft distal end configured to receive a fluid;
an electrode assembly extending distally from the shaft distal end, the electrode assembly defining a distally located central hub portion and a plurality of splines each including a distal end portion extending from the central hub portion, and a proximal end portion attached to and constrained by the outer shaft, the plurality of splines forming an inner space, the electrode assembly comprising:
a flexible circuit having a flex circuit hub and a plurality of flex circuit branches extending proximally from the flex circuit hub, the flexible circuit further including an ablation electrode including an ablation electrode hub portion located on the flex circuit hub and a plurality of radial segments integrally formed with the ablation electrode hub portion, each of the radial segments extending proximally along a portion of a respective one of the flex circuit branches and terminating in a proximal end; and
a central post extending from the shaft distal end into the inner space having a post electrode and a fluid port disposed in the inner space, the fluid port in fluid communication with the fluid tube and configured to disperse the fluid into the inner space, the central post proximal to the proximal end of the ablation electrode, wherein the post electrode is one of a planar electrode or a bulbous electrode.

20. The catheter of claim 19, wherein the post electrode is a bulbous electrode, and the bulbous electrode is inflatable.
